(19) United States
(12) Patent Application Publication
(10) Pub. No.: US 2005/0054025 A1
(43) Pub. Date: Mar. 10, 2005

(54) Stabilized enzymes for detecting and monitoring chemical toxins

(76) Inventors: Kim R. Rogers, Henderson, NV (US); Howard H. Weetall, Las Vegas, NV (US)

Correspondence Address: BROWDY AND NEIMARK, P.L.L.C.;624 NINTH STREET, NW, SUITE 300, WASHINGTON, DC, 20001-5303, US

(21) Appl. No.: 10/657,249

(22) Filed: Sep. 9, 2003

(51) Int. Cl. C12Q 1/46; C12M 1/34

(52) U.S. Cl. 435/20; 435/287.1

(57) ABSTRACT

Organophsophorus or carbamate compounds can be detected using the enzyme acetylcholinesterase for which these compounds are inhibitors wherein the enzyme is immobilized in a sol-gel or in a membrane.

### FIELD OF THE INVENTION

The present invention relates to methods and apparatus for detecting hazardous chemicals in the environment.

### BACKGROUND OF THE INVENTION

Organophosphorus and carbamate compounds (hereinafter also referred to as OP compounds) are used extensively in insecticides. Although these compounds are effective as pesticides, they are among the most toxic products produced by the chemical industry, and are highly toxic to many organisms, including humans. Insecticide residues are found in soil and groundwater, and the detection of these residues is important so that they can be eliminated from the environment, as well as to protect the health of both humans and animals. OP compounds are also used in nerve agents such as sarin, soman, and tabun, for chemical warfare purposes.

OP compounds are some of the most potent toxic agents; they are specific inhibitors of acetylcholinesterase. The result of acetylcholinesterase poisoning is a cholinergic crisis in humans; the clinical effects are directly related to accumulation of the inhibitors. Nerve agents are classified into G agents (GD, soman; GB, sarin; or GA, tabun) and V agents (VX). These agents differ from one another in physical properties. For example, VX has a much lower vapor pressure than the G agents. However, the toxicity and main effects of these compounds are similar-inhibition of acetylcholinesterase and subsequent breakdown of the normal operation of the autonomic and central nervous systems. Since OP compounds can be used in agriculture by relatively unsophisticated workers, or can possibly be used by terrorists as chemical warfare agents, it is extremely important to be able to detect OP compounds rapidly and reliably to prevent casualties from OP exposure.

Because many pesticides and nerve agents inhibit acetylcholinesterase, this enzyme has been used in many forms as a detector molecule for these agents by a variety of techniques (1-35). These methods typically use one of two formats, in which the immobilized enzyme is pre-incubated with the inhibitor prior to addition of substrate or brought into contact with the enzyme along with the substrate. In either case, the majority of these methods require some form of instrumentation. Detection methods used for analysis have included optical methods, electrochemical methods, and some form of flow-through system or chromatography.

Some of the sophisticated instrumental methods developed for determining cholinesterase inhibitors involve the use of gas and liquid chromatography and mass spectrometry. Additionally, a number of liquid phase chemiluminescence procedures have been developed to detect organic species, mostly using the luminol and peroxyoxalate reactions, as described in Robards et al., Anal. Chem. Acta 266:147, 1992.

These traditional methods are not practical for individual use, as the methods are time consuming and expensive, the apparatus is expensive and not portable, and requires high maintenance. Additionally, measurement of OP compounds in mixtures using these traditional methods requires cumbersome extraction and manipulation procedures.

Biosensors have been developed as an alternative to the traditional gas and liquid chromatography and mass spectrometry technology. Generally, biosensors include those which are enzyme-based and bioaffinity-based. An enzymatic biosensor uses an enzymatic or metabolic process to detect a reaction product which occurs between an incoming substrate and an immobilized enzyme. A bioaffinity sensor relies on a biological binding event of a target substance.

Previously known biosensors are electronic devices that produce electronic signals as the result of biological interactions. These biosensors comprise a biological receptor linked to a transducer, such as an electronic, optical, or acoustic transducer, in such a way that biochemical activity is converted into electrical activity. The electronic component of the biosensors measure voltage, amperage, wavelengths, temperature, conductivity or mass.

Biosensors are widely used to detect biological pharmacological, or clinically important compounds. Generally, enzyme biosensors are selective, sensitive, and specific. They are portable, simple, and easy to use. Enzymatic biosensors can detect only those substances of interest and ignore all other environmental and biological interference.

Various cholinesterase biosensors have been developed. These biosensors comprise cholinesterases covalently or non-covalently immobilized on a support, including covalent bonding, entrapment, adsorption, copolymerization, ionic bonding, and cross linking. Cholinesterases have been immobilized on a wide variety of solid and gel supports such as glass, silica, ion-exchange resins, agarose, and nylon supports. Ideally, the preferred methods of immobilizing enzymes on solid supports have high coupling rates, and the preferred biosensors retain enzymatic activity and maintain stability. However, some biosensors which have non-covalently bound enzyme possess undesirable characteristics, such as enzymatic instability at ambient and/or denaturing conditions, a propensity of the enzymes to leach from the surface to which it was non-covalently bound, and a short half-life in solution.

### SUMMARY OF THE INVENTION

It is an object of the present invention to overcome the aforesaid deficiencies in the prior art.

It is another object of the invention to provide detectors for long-term detection of organophosphate and carbamate compounds.

It is a further object of the present invention to entrap enzymes in a sol-gel glass for detection of organophosphate and carbamate compounds.

It is yet another object of the present invention to entrap enzymes in a membrane for detection of organophosphate and carbamate compounds.

The present invention provides a method and apparatus for detecting organophosphate and carbamate compounds that is inexpensive, rapid, sensitive, and that can be used with or without read-out instrumentation.

According to the present invention, acetyl cholinesterase is immobilized in a sol-gel or a membrane as an adduct in order to detect the presence of organophosphate and carbamate compounds. If any of these compounds are in the environment in which the enzyme is located, the inhibitors will inhibit the enzyme and an indicator present in the adduct will indicate the presence of the organophosphate and carbamate compound.

For acetylcholinesterase to be used successfully over a long period of time, the enzyme must be in a form that has long-term stability at a variety of temperatures and under a variety of adverse conditions. It was discovered that, by immobilizing the enzyme in a sol-gel or in a membrane, the enzyme was highly stable over a range of temperatures and in a variety of conditions.

The present invention provides improved methods for immobilizing acetylcholinesterase that can be used in detectors for detecting organophosphate and carbamate compounds.

In one embodiment of the present invention, the enzyme is immobilized in a sol-gel. In another embodiment of the present invention, the enzyme is immobilized in a membrane.

According to the present invention, a sample of the material treated with an inhibitor is compared with a sample of the material, either sol-gel or membrane, that is not treated with inhibitor. The assay method produces a color that can easily be distinguished in a test-tube-type assay without the requirement for an instrument, or in a 96-well microplate using an absorbance reader. The time to complete the assay is anywhere from about five to 60 minutes, depending upon the quantity of adduct used.

Any type of reagent that develops a visible color can be used in the assay of the present invention. The preferred reagents are Ellman's reagent, 5,5-dithiobis(2-nitrobenzoic acid) and acetylthiocholine iodide.

### BRIEF DESCRIPTION OF THE DRAWINGS

FIG. 1 shows the inhibition curve for disopropylfluorophosphate (DFP) in solution. The curve was generated from a sigmoidal fit and the IC₅₀ (concentration yielding 50% inhibition) was graphically determined to be 700 ppb.

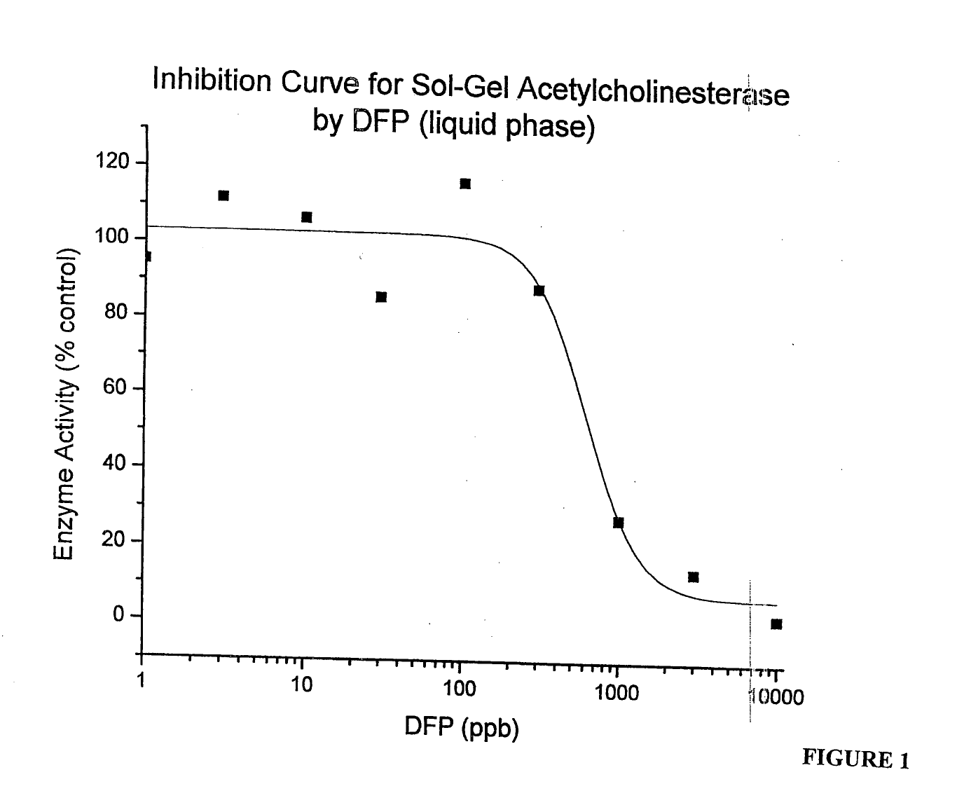

FIG. 2 shows the inhibition profile for DFP vapor. Concentrations were determined by volume, assuming complete vaporization of the DFP. The methanol control showed little effect on the acetylcholinesterase activity. The inhibition data were fit using a sigmoidal fit, and the IC₅₀ was determined graphically to be 1.2 ppm.

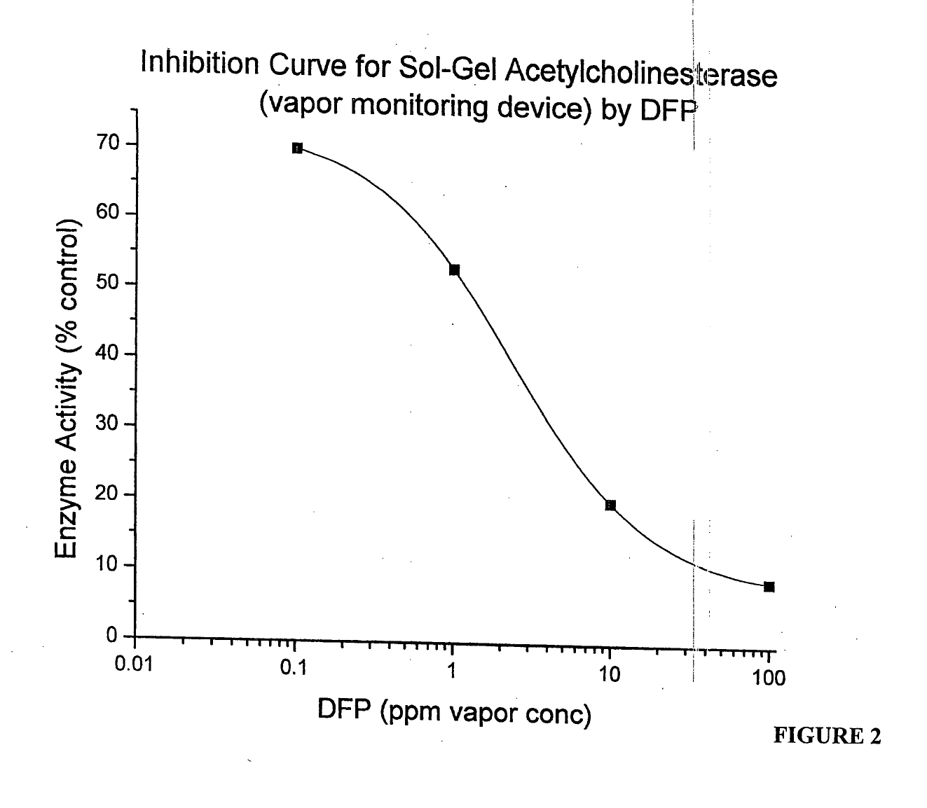

FIG. 3 shows a photograph of the DFP inhibited sampling devices containing the sol-gel acetylcholinesterase and Elman reagent. The devices correspond to data points in FIG. 2. This illustrates the feasibility for a simple color change test to be applied to detection of a nerve agent surrogate in vapor form.

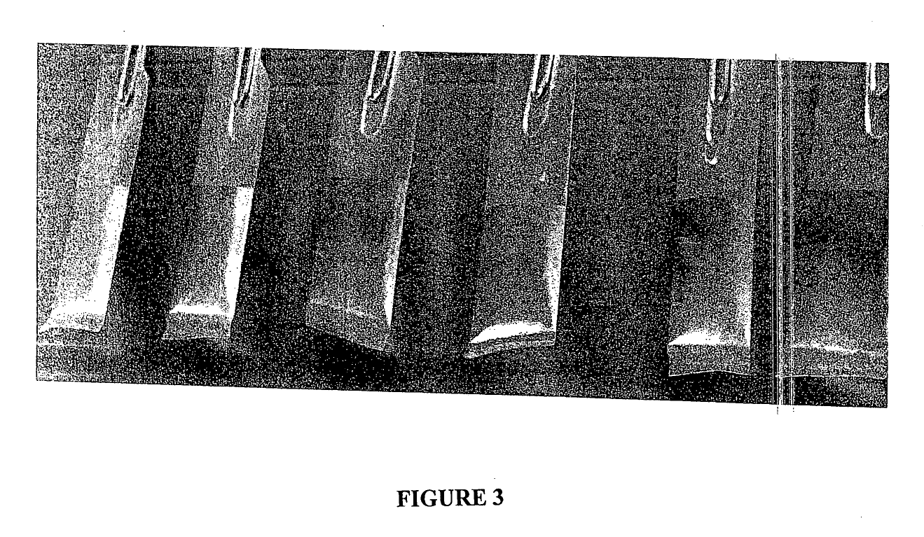

FIG. 4 shows a design for an example of a self-contained anticholinesterase sampling device.

FIG. 5 shows the storage stability of sol-gel acetylcholinesterase at room temperatures. Samples were ground but not sieved. Samples were weighed out and assayed as described in the Detailed Description of the Invention.

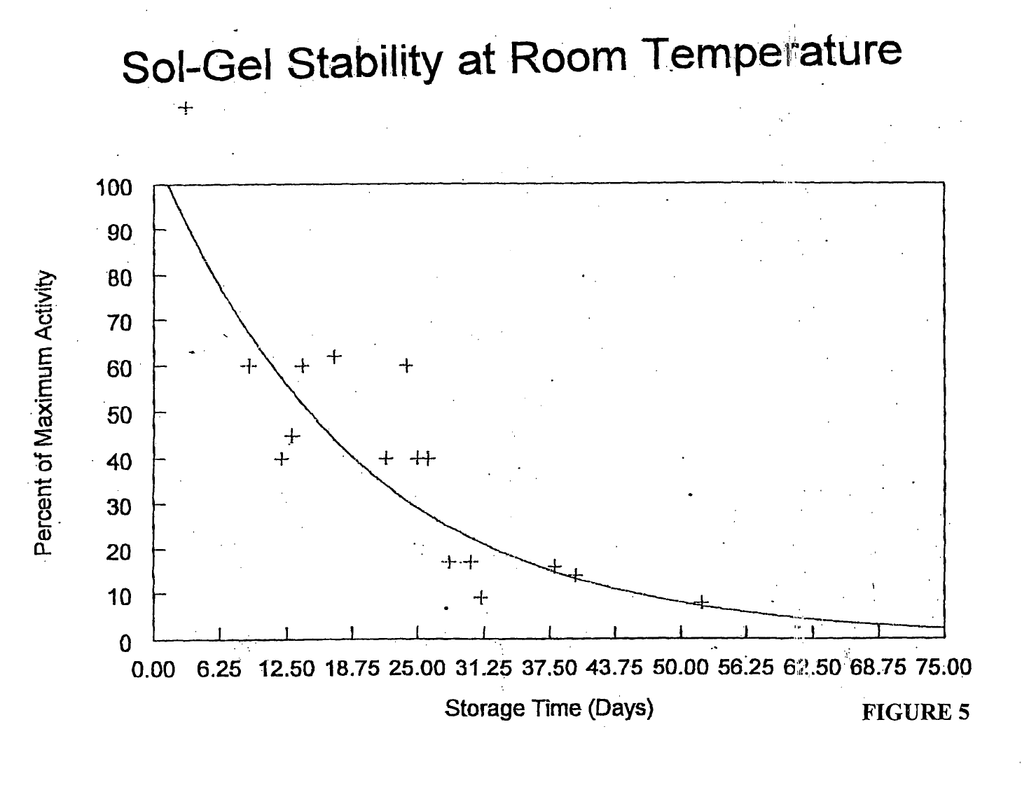

FIG. 6 illustrates the thermal stability of a dry sample of unsieved sol-gel acetylcholinesterase at 75° C., maintained in an oven.

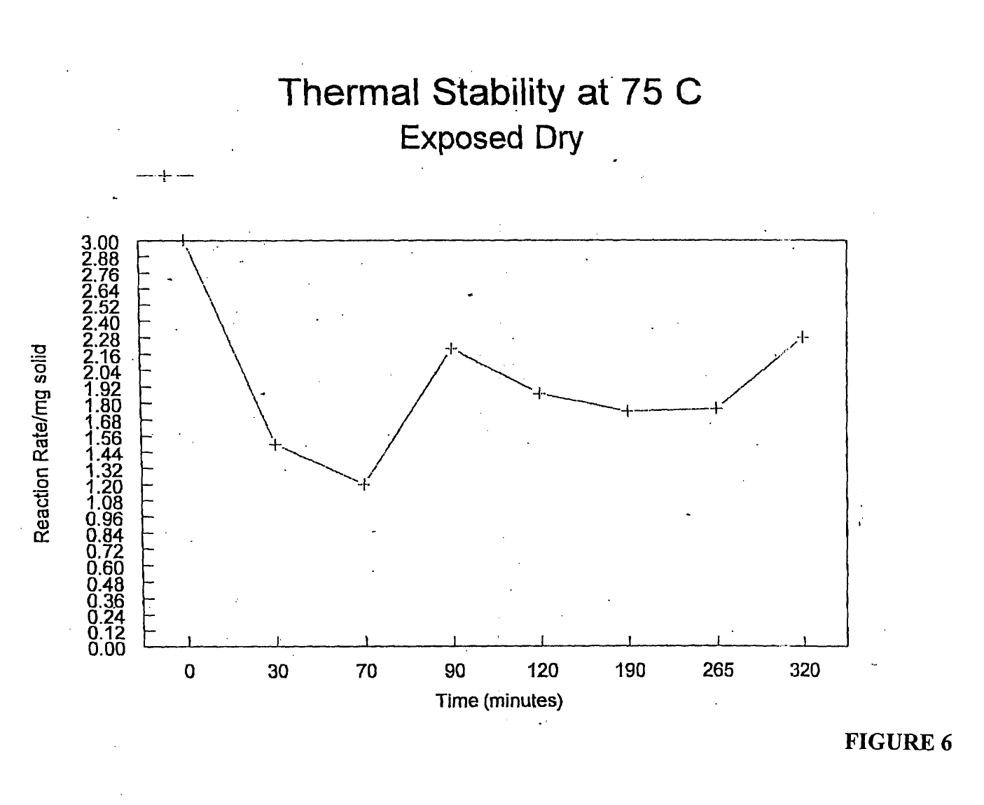

FIG. 7 shows the thermal stability of a dry sample of unsieved sol-gel acetylcholinesterase at 100° C. maintained in an oven.

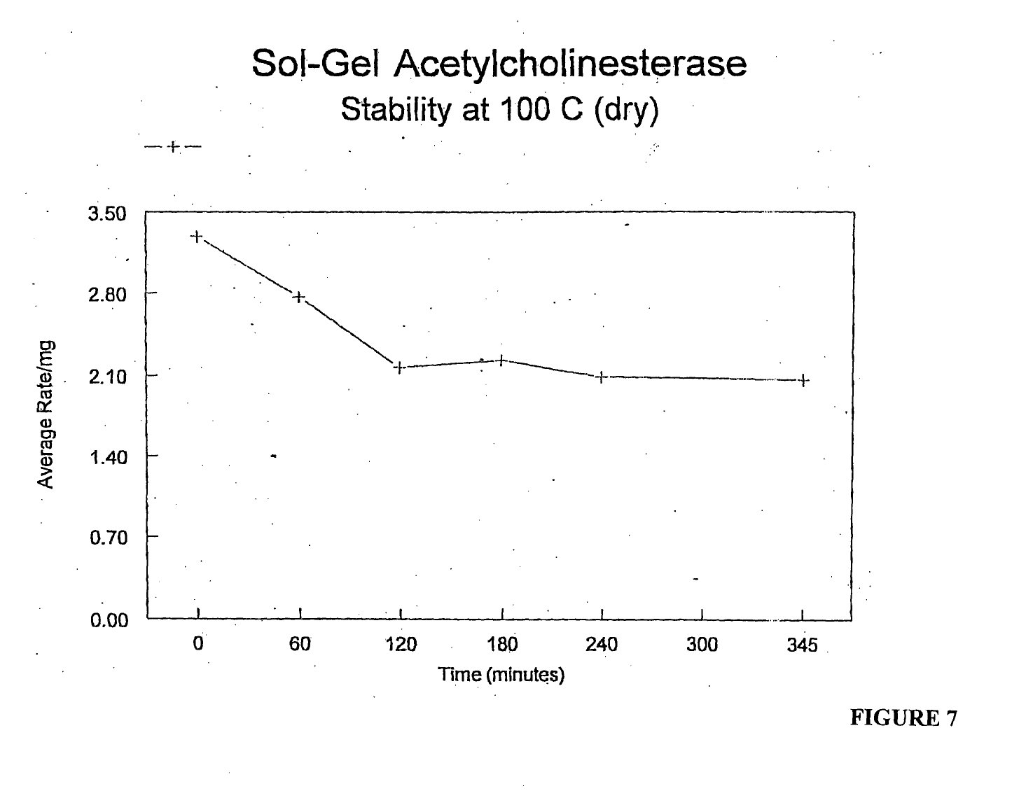

FIG. 8 shows the stability of +140 U.S. mesh and +400 U.S. mesh sol-gel acetylcholinesterase samples heated in a water bath at 80° C. Samples were suspended in 0.05 M Na₂HPO₄ solution during the heating. Samples were removed at intervals over several hours and assayed.

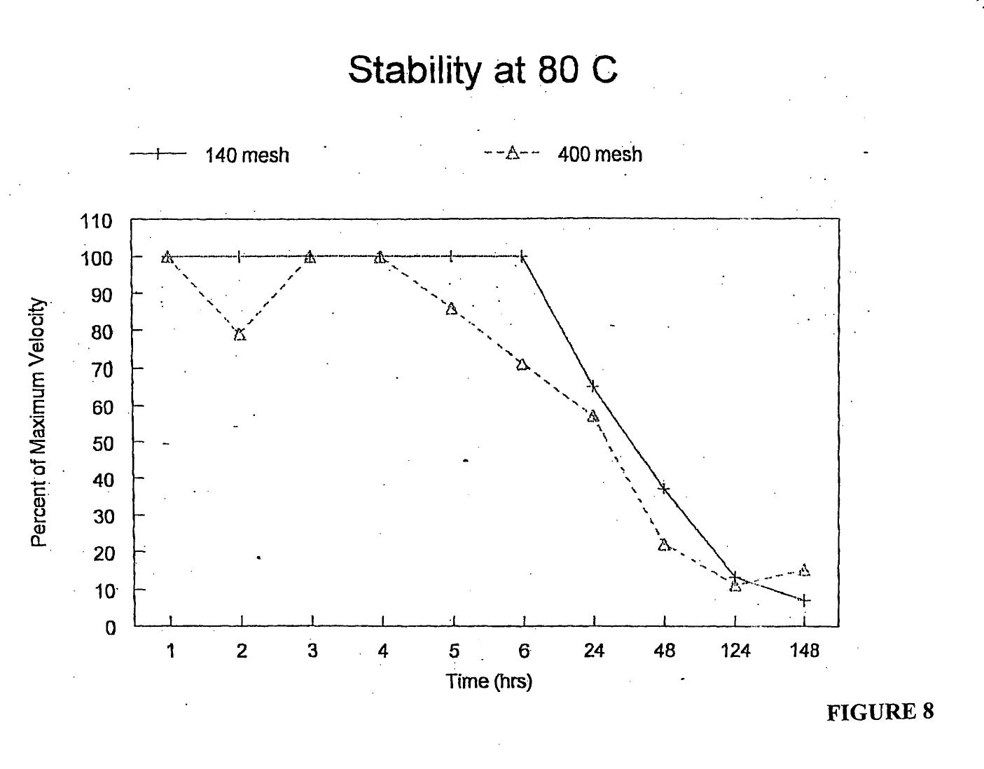

FIG. 9 shows the pH profiles of the soluble and sol-gel forms of acetylcholinesterase. The sol-gel was not sieved. The optimum for the immobilized form has shifted slightly to the acid side. However, at pH 6, the soluble form is inactive, while the immobilized form retains 60% of its maximum activity.

FIG. 10 shows the activity of sol-gel acetylcholinesterase vs. particle size. Between +140 mesh and +400 mesh there is an increase in activity of approximately 8-fold.

### DETAILED DESCRIPTION OF THE INVENTION

The present invention provides methods and apparatus for long term detection of organophosphate and carbamate pesticides and nerve agents that are inhibitors of cholinesterase.

The following nonlimiting examples illustrate the method of the present invention.

Sel-Gel Immobilization

### EXAMPLE 1

Acetylcholinesterase was entrapped in a sol-gel glass prepared from tetramethylorthosilicate (TMOS) using the method described in Weetall, “Retention of bacteriorhodopsin activity in dried sol-gel glass,” Biosensors and Bioelectronics 11: 327-333, 1996. The enzyme was added to 25 mL of a sugar solution, which stabilized the enzyme. To this was added a solution of 7.0 mL of TMOS plus 3.0 mL of distilled water plus 0.05 mL of 0.04 M HCl that was previously prepared and shaken for 30 minutes.

The sugar solution contained the following ingredients:
5% trehalose
10% glucose
0.1% gelatin
0.02% sodium azide
1% NaCl.

Aliquots of the mixture were prepared and allowed to gel, followed by drying and either grinding the gel into small particles or retaining the gel in 2.5 mL cuvettes or 10 mm×75 mm test tubes to form blocks. All samples were stored at room temperature and exposed to the laboratory atmosphere during the drying process.

In this example, the sugar, trehalose, was chosen because it is a well known stabilizer for proteins. However, other sugars that stabilize proteins, such as maltose, can be used. Additionally, the gelatin was included as an aid in stabilizing the enzyme, and sodium azide was added as a preservative.

These materials were tested weekly for remaining activity using a standard method for quantitating this enzyme. Additionally, these materials were tested for inhibition by addition of known organophosphates both in solution and as a vapor.

For use in long term detection and quantitation of organophosphate and carbamate compounds, the gel or membrane containing the enzyme was inserted into a length of tubing, such as dialysis tubing. In addition, a sample of the same material was placed into the same container but barrier packaged to prevent contact with the inhibitor, but not with the fluctuations in temperature to which the adduct was subjected during the test period. The immobilized enzyme was removed and assayed for activity vs. the barrier packaged sample to determine the loss in activity due to exposure to the environment.

Experimental Data: Samples were examined spectrophotometrically at 409 nm. Enzyme activity was measured using the Ellman method [Ellman et al., Biochem. Pharmacol (1961) 7: 88-95].

Experiments in Aqueous Solution

Paraoxon diluted 1:1,000,000 in hexane was added to a series of test tubes over a dilution range of 10⁻⁶ to 10⁻¹⁴. Each tube contained 10 mg of sol-gel glass containing enzyme in 0.33 mL of 0.005M buffer. Assay reagents brought the volume to 1.0 mL.

This experiment was repeated for a total of three times, and exhibited a range of paraoxon detection of from 12 ppb to as low as 1 ppb, depending upon the time allowed for incubation with the inhibitor and the size of the particles of the sol-gel. As was expected, the length of time for incubation was directly related to a degree of inhibition. The particle size of the ground sol-gel can range from about 100 U.S. mesh to about +400 U.S. mesh size.

It is clear that the incubation time, the particle size (ability of the inhibitor to reach the enzyme) and the enzyme and inhibitor concentrations are all interdependent, and there are optimum sets of conditions depending upon the environment and time required for the exposure of the agents. These optimum times can be determined using the methods described above by one skilled in the art without undue experimentation. The above data demonstrate that the product produced is stabilized over time and is inhibited by a standard acetylcholinesterase inhibitor, making this material an excellent choice for long term exposure to conditions in homes, offices, and public places for detecting cholinesterase inhibitors.

Further Inhibition Study of Acetylcholinesterase in Sol-Gel Glass

To prepare the adduct, 3.0 mL distilled water plus 0.05 mL of 0.04M HCl was added to 7.0 mL TMOS. This was shaken for 30 minutes at room temperature and the volume was increased by 40 mL by adding 5.0 mL of 0.1M Na₂HPO₄ plus 35 mL of a sugar solution made up of 5% trehalose, 10% glucose, 0.1% gelatin, 0.02% azide and 1.0% NaCl. To this solution was quickly added 33 units of acetylcholinesterase (Sigma, St. Louis, Mo.). After gelation and drying, the material was ground into small particles and used for assay by the Ellman method.

Inhibition Study

The inhibition study using microtiter plates was conducted at room temperature. The assay was for 25 minutes using 0.015 mL of paraoxon serially diluted 10-fold in the buffer plus 0.075 mL 5,5′-dithio-bis(2-nitrobenzoic acid) at 1 mM and 0.075 mL of acetylcholine iodide at 1 mM. The samples were preincubated with the paraoxon for 60 minutes prior to the assay.

Paraoxon (0.1 mL) was diluted in buffer using 10-fold serial dilutions. An aliquot of 0.1 mL was removed from each dilution of diluted paraoxon above and placed into a well of a microtiter plate. To each well was added adduct prepared with 33 units or 333 units of enzyme. This was allowed to incubate for 60 minutes prior to assay. The paraoxon dilution giving 50% inhibition per mg of adduct was calculated both in parts per million and molar concentration of diluted paraoxon.

| | | 50% inhibition (Paraoxon |
|---|---|---|
| Adduct | 50% inhibition (ppm) | concentration) |
| 333 units added | 0.01 | 3.3 × 10⁻⁹ M |
| 33 united added | 0.005 | 1.65 × 10⁻⁹ M |

Activity Recovery

The Ellman assay was used to determine activity vs the amount of enzyme immobilized. A known amount of adduct was added to a 1.0 mL cuvette, followed by the substrate solutions, and assayed for 5-25 minutes, depending upon the activity. Recovery was determined based on assays carried out with known quantities of soluble enzyme. Studies were conducted using 333 units and 33 units added to the prepared TMOS adducts.

Adduct (% activity recovered) for 3.3 g adduct total

| | |
|---|---|
| 333 units added | 40% recovery |
| 33 units added | 27% recovery |

Determination of Optimum pH

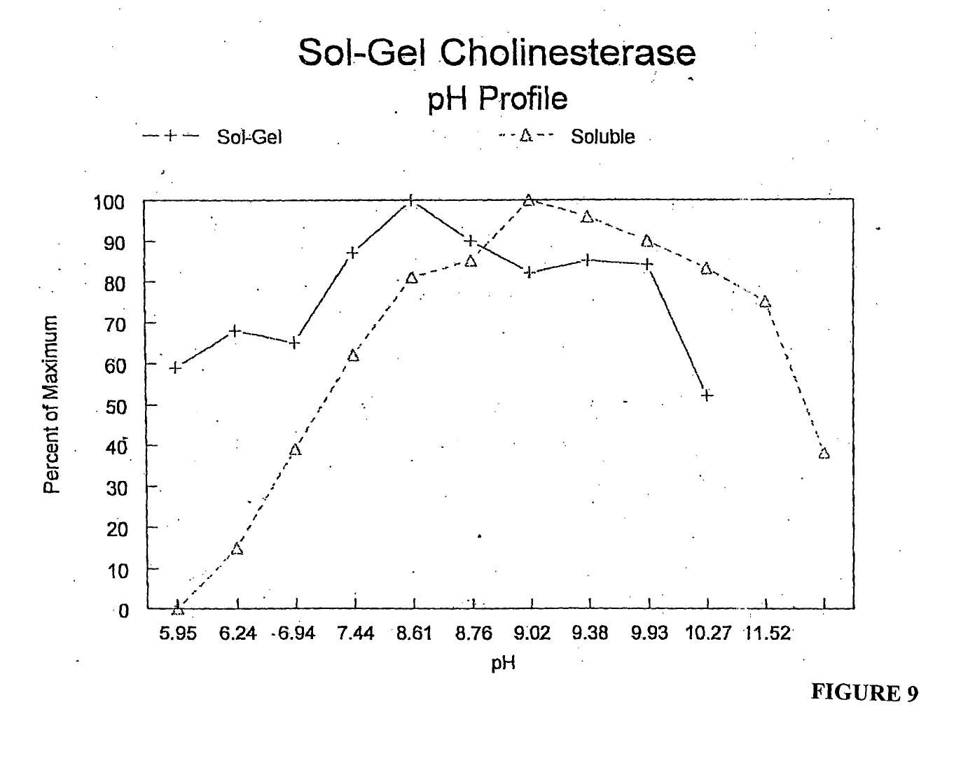

Both soluble and immobilized acetylcholinesterase were assayed to obtain pH profiles. Samples of the immobilized enzyme were weighed out in triplicate and placed into microtiter wells with 0.75 mL of 0.05 M sodium phosphate buffer previously adjusted to the desired pH by mixing monobasic with dibasic sodium phosphate and, when necessary, adding NaOH to adjust the solution to the desired pH. The assay reagents were added to each adduct, and the adducts were assayed kinetically. Results were obtained and the rate of change in OD was divided by the sample weight to normalize the results. Since all adducts were assayed over the same time period, it was unnecessary to further normalize the values. The results are shown in FIG. 9.

Development of Km

A two-fold series of substrate dilutions was prepared. Into each microtiter well was placed 0.075 mL of each component of the assay reagent. A 0.10 mL sample of soluble enzyme containing 10 units of activity was diluted 1:100 twice to a final dilution of 1:10,000 and 0.075 mL added to each of the microtiter wells. The assay was conducted in the kinetic mode.

In the case of immobilized enzyme, the adduct samples were first weighed out and placed into the microtiter wells. To each well was added 0.075 mL of 0.05 M Na₂HPO₄ followed by the assay reagents. The assay was conducted in the kinetic mode and the resulting changes in OD were divided by the sample weights to normalize the results.

Because the assay times were the same for all samples, no further normalization was required. The results were as follows: Km for soluble enzyme was 1.18×10⁻⁶M. Km for enzyme immobilized in sol-gel was 133×10⁻⁶M.

Activity vs. Mesh Size

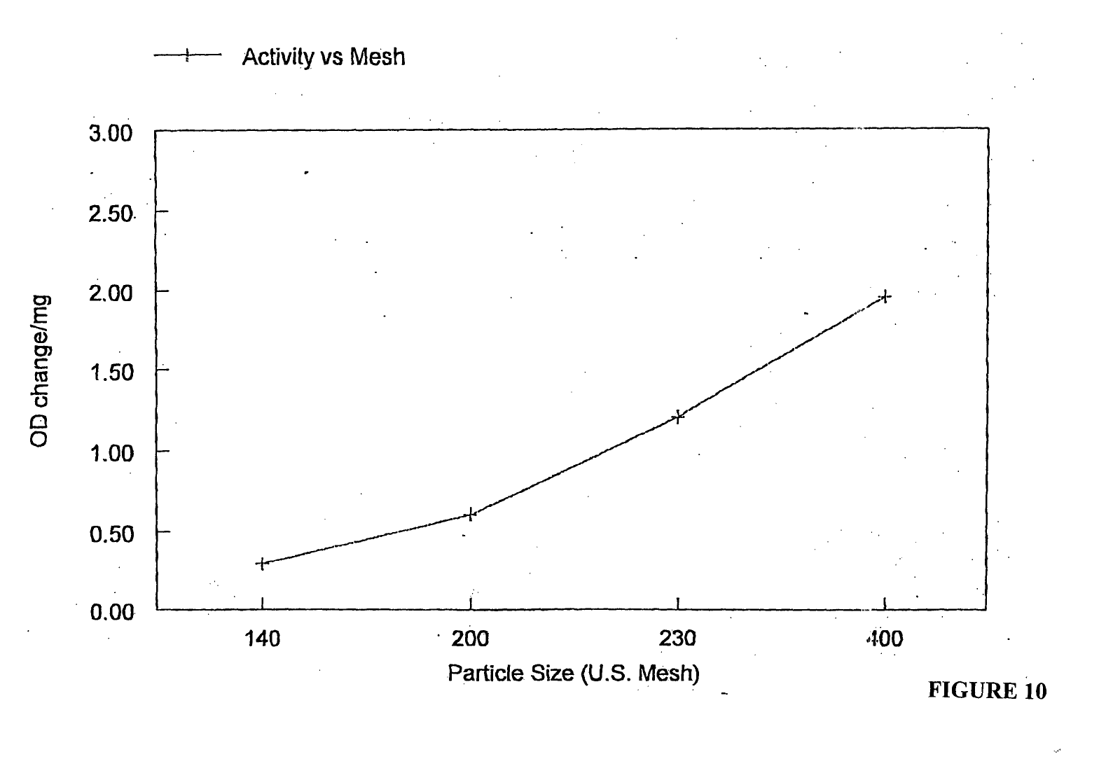

Several samples of the enzyme adduct prepared at different times were combined and ground in a mortar and pestle for several minutes. The resulting material was sieved through a series of screens into particle ranges as follows: +140 mesh, 140-200 mesh, 200-230 mesh, 230-400 mesh and smaller than 400 mesh. The separated materials were assayed in triplicate for activity in microtiter plates by first weighing the samples, adding 0.075 mL of 0.05 M Na₂HPO₄, followed by the assay reagents. The assays were carried out kinetically. The resulting change in OD was divided by the sample weight to normalize the results. The results are shown in FIG. 10.

Experiments in Vapor Phase

Vapor phase experiments were conducted by adding acetylcholinesterase sol-gel adduct (10 mg) to semipermeable polyethylene tubing (3 in) and heat sealing both ends. These sampling devices were then placed into 40 mL glass vials at final (vol/vol) dilutions of DFP ranging from 0.1 to 100 ppm. After 12 hr the samples were removed, opened, and the enzyme activity determined by adding the Ellman reagents directly to the sampler. After 30 minutes the enzyme activity was determined using absorbance at 340 nm with a portable spectrophotometer.

Membrane Immobilization

Membranes that can be used in the detectors of the present invention include any membrane that can bind protein. While the illustrations in the present application all refer to POREX® membranes, membranes made of nylon, cellulose acetate, cellulose nitrate, or the like can also be used successfully as detectors.

For the following examples, all chemicals were reagent grade. Paraoxon was purchased from Sigma-Aldrich (Milwaukee, Wis.). The organophosphates and carbamates were purchased from Chemical Services (West Chester, Pa.), and all contained the pesticide at a concentration of 100 μg/mL. A commercially available kit was used for comparison with the “Dot Assay” described herein. The lateral flow strips were prepared from a sheet of POREX® LATERAL-FLO™ membrane materials (Porex Corp., Fairburn, Ga.) Each strip was approximately 0.25 inches×2.0 inches.

The solution used to prepare the activated strips contained 5% trehalose, 10% glucose, 0.1% gelatin, 0.02% azide, and 1% NaCl dissolved in 0.2 M Na₂HPO₄. A range of 10 to 300 units of acetylcholinesterase was added to the sugar solution. The strips were immersed in the enzyme containing solution for approximately five to 30 minutes, removed, and allowed to dry. All samples were stored at room temperature and exposed to the laboratory atmosphere or at 4° C. For stability studies only, samples stored at room temperature were tested because these samples would be the most susceptible to activity loss vs. time as compared with the refrigerated samples. The studies reported below were accomplished with batch wise prepared strips that had been stored at 4° C. over a period of approximately one month.

Solution Inhibition Assay Method

All of the organophosphate pesticides, except the paraoxon, were diluted into distilled water to a concentration of 10 μg/mL, and were oxidized by addition of 1% bromine solution. The pesticide was allowed to oxidize for 20 minutes, followed by addition of 0.012 mL of ethanol used to stop the reaction.

Assays were performed in test tubes using ten-fold serial dilutions of the pesticide into 0.100 mL of 0.05 M Na₂HPO₄ solution to which had been added small 5 mm circular dots punched from the strips with a ticket punch. The enzyme-containing dots were allowed to stand for 20 minutes before addition of substrate.

Each tube received 0.10 mL of 1 mM 5,5-dithio-2-bis-nitrobenzoic acid (DTNB) and 0.10 mL of 1 mM acetylthiocholine iodide. For the visual determination, after ten minutes the tube in the dilution series showing less color than the control was noted. The resultant solutions were then transferred into microtiter plates where they were read in a standard plate reader using the endpoint mode with dual wavelengths of 405 nm and 490 nm for background.

To determine the IC₅₀ and IC₂₀ values for a series of organophosphates and carbamates, the data were plotted on a semi-log scale and values were determined directly. Paraoxon was also examined for inhibition. In this case, the first tube containing paraoxon was initially diluted to a dilution of 10⁻⁵ followed by 10-fold serial dilutions into test tubes containing 0.10 mL of 0.05 M Na₂HPO₄ before addition of the 5 mm POREX® dot. The samples were allowed to incubate for 20 minutes before addition of another 0.10 mL of each of the assay reagents. The results were determined visually with detectable levels of 10 ng/mL.

It was found that the assay of the present invention is able to detect amounts of as little as about 0.5 ng/mL of paraoxon using instrument detection.

Stability Testing

A sample of membrane materials as prepared above were tested weekly for remaining enzyme activity. The enzyme was immobilized in 5 mm dots obtained by a ticket punch. The activity was determined using a standard microtiter plate and plate reader. The assay solutions contained 0.075 mL Na₂HPO₄ buffer and 0.075 mL of each of the assay reagents. The assays were run over a period of six minutes, with readings taken every 14 seconds.

Upon immobilization, the acetylcholinesterase retained 31% of its original activity (as measured in solution). After the initial loss in activity, however, the enzyme was quite stable. The immobilized enzyme stored at room temperature in the laboratory showed an exponential rate of decay, and over the 95 days of storage retained 16% of the starting activity. Samples stored at refrigerated temperature lost less than 10% of the initial activity over the approximately 30 days during which the inhibition experiments were performed.

Inhibition with Oganophosphate and Carbamate Insecticides

The organophosphate and the carbamate pesticides were assayed using the above-described 5 mm dots and were compared to results using a commercial kit purchased from Envirologix. The results shown in Table 1 indicate that the dot assay showed similar sensitivity to the commercially available kit for most of the samples tested. In a few cases, comparisons could not be made because the particular pesticide was not listed in the kit's package insert.

TABLE 1
Porex Dot Test

| | | | | |
|---|---|---|---|---|
| | Porex Dot Test | EnviroLogix | MCGL | HA |
| Agent | IC20 (μg/L) | IC₂₀(μg/L) | (μg/L) | (μg/L) |

Organophosphates

| | | | | |
|---|---|---|---|---|
| Parathion (methyl) | 2 | 63 | — | 2 |
| Malithion | 0.11 | 60 | — | 100 |
| Diazinon | 0.14 | 3.0* | — | 0.6 |
| Chloropyrifos | 0.1* | 5.2 | — | 20 |
| Disulfoton | 0.5 | ND | — | 0.3 |
| Terbufos | 143 | 12* | — | 0.9 |

Carbamates

| | | | | |
|---|---|---|---|---|
| Oxymyl | 16.7 | 100 | 20 | 20 |
| Carbaryl | 9.0 | 110 | — | 700 |
| Carbofuran | 0.4 | 40 | 40 | 10** |
| Aldicarb | 33 | ND | — | 7 |
| Methomyl | 0.011 | ND | — | 200 |
| Baygon (propoxur) | 9 | 50 | — | 3 |

*IC₅₀ values
**Anticipated revised value for both the 1991 MCLG and the 1988 HA.
NOTE:
the above MCLG and HA values are excerpted from the drinking water table, Summer 2002 at: http://www.epa.gov/waterscience/drinking/standards/dwstandards.pdf

Detection Devices

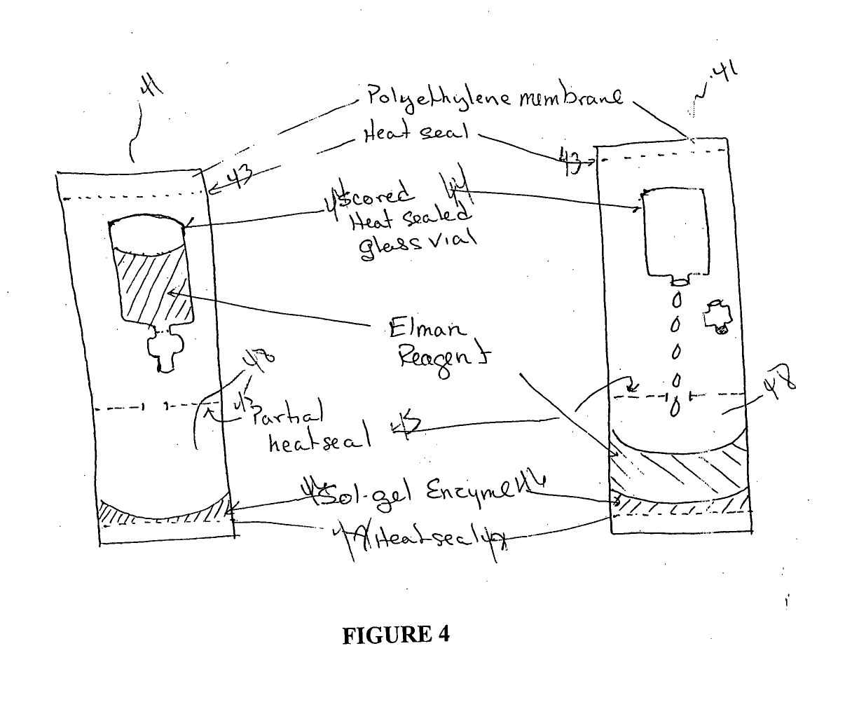

The immobilized enzymes can be incorporated in any type of holder or package that permits exposure of the immobilized enzyme to the atmosphere at the time testing of the atmosphere is to commence. One example of such a device is shown in FIG. 4.

To use the device shown in FIG. 4, one exposes the device to a chemical agent sought to be detected. The scored vial 44 is broken inside the sealed device 41 and liquid reagent is permitted to drain into the lower compartment of the device 48. The device is incubated for about 30 minutes, after which the color of the assay reagent in the device is compared to that of a blank (non-exposed device).

Thus, the present invention provides methods for detecting inhibitors of acetylcholinesterase down to concentrations of 10 ng/mL without the use of any instrumentation. This detection limit is competitive with many instrumental methods. In addition, assays using the immobilized enzymes of the present invention are more rapid, less expensive, and require less expertise than conventional assays for organophosphates and carbamates. While the assays can be conducted in test tubes with a visually determined endpoint, instrumental detection can also be used. Instrumental detection allows detection of amount as low as 0.1 ng/mL.

The foregoing description of the specific embodiments will so fully reveal the general nature of the invention that others can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out various disclosed functions may take a variety of alternative forms without departing from the invention.

Thus, the expressions “means to . . . ” and “means for . . . ” as may be found in the specification above and/or in the claims below, followed by a functional statement, are intended to define and cover whatever structural, physical, chemical, or electrical element or structures which may now or in the future exist for carrying out the recited function, whether or nor precisely equivalent to the embodiment or embodiments disclosed in the specification above. It is intended that such expressions be given their broadest interpretation.

1. A detector for detecting at least one organophosphorus or carbamate compound comprising the enzyme acetylcholinesterase immobilized in a sol-gel or a membrane, wherein the enzyme is inhibited by at least one of the organophosphorus or carbamate compounds.

2. A method for detecting at least one organophosphorus or carbamate compound in a sample comprising contacting said sample with enzyme acetylcholinesterase immobilized in a sol-gel or a membrane, wherein the enzyme is inhibited by at least one of the organophosphorus or carbamate compounds.

3. The method according to claim 2 wherein the sample is contacted with acetylcholinesterase immobilized in a sol-gel or a membrane wherein the pH ranges from about 5.95 to about 11.52.

4. The method according to claim 2 wherein the compound detected is an organophosphorus compound and 1% bromine is added to the organophosphorus compound prior to addition to the immobilized enzyme.

5. The method according to claim 2 wherein the enzyme is immobilized in a sol-gel.

6. The method according to claim 2 wherein the enzyme is immobilized in a membrane.

7. A detector for detecting at least one compound selected from the group consisting of organophosphorus and carbamate compounds which are inhibitors for the enzyme acetylcholinesterase, wherein the actcylcholinesterase is immobilized in a sol-gel or in a membrane, wherein said sol-gel or said membrane is packaged so that when a test is conducted the enzyme is exposed to ambient conditions.

8. The detector according to claim 7 wherein the enzyme is immobilized in a sol-gel.

9. The detector according to claim 7 wherein the enzyme is immobilized in a membrane.

10. The detector according to claim 7 wherein the package comprises a semipermeable polyethylene bag which is opened after exposure of the enzyme to inhibitor to commence the enzyme assay.
